(19) United States
(12) Patent Application Publication
(10) Pub. No.: US 2024/0052433 A1
(43) Pub. Date: Feb. 15, 2024

(54) DEVICE AND ASSAY FOR DIAGNOSING TUBERCULOSIS AND ASSOCIATED ANTIBIOTIC RESISTANCES

(71) Applicant: Formulatrix International Holding Ltd., Dubai (AE)

(72) Inventors: Kabir Yamana, Bedford, MA (US); Michael Nilsson, Bedford, MA (US); Sean Dineen, Bedford, MA (US); Rasmus Lindblom, Bedford, MA (US); Kyle Hallock, Bedford, MA (US)

(73) Assignee: Formulatrix International Holding Ltd., Dubai (AE)

(21) Appl. No.: 18/383,270

(22) Filed: Oct. 24, 2023

Related U.S. Application Data
(63) Continuation-in-part of application No. 18/032,455, filed on Apr. 18, 2023.
Application No. 18/032,455 is a continuation-in-part of application No. 18/032,457, filed on Apr. 18, 2023.
Application No. 18/032,457 is a continuation-in-part of application No. 18/032,459, filed on Apr. 18, 2023.
Application No. 18/032,459 is a continuation-in-part of application No. 18/032,463, filed on Apr. 18, 2023.
(60) Provisional application No. 63/093,640, filed on Oct. 19, 2020.
(60) Provisional application No. 63/093,640, filed on Oct. 19, 2020.
(60) Provisional application No. 63/093,640, filed on Oct. 19, 2020.
(60) Provisional application No. 63/093,640, filed on Oct. 19, 2020.

(51) Int. Cl. C12Q 1/689; C12Q 1/6851; B01L 3/00; B01L 7/00

(52) U.S. Cl. CPC C12Q 1/689; C12Q 1/6851; B01L 3/502715; B01L 7/52; C12Q 2600/106; B01L 2200/16; B01L 2300/0654; B01L 2200/04; B01L 2300/0883; B01L 2300/18

(57) ABSTRACT

In some aspects, a system is disclosed for detecting genetic targets for diagnosing tuberculosis and associated antibiotic resistance. The system includes a mechanical instrument and a cartridge assembly. The cartridge assembly comprises one or more reservoirs, with at least one reservoir containing a wet reagent. The cartridge also includes a chip with a fluidic channel holding a liquid slug. The chip has an optical detection region positioned between a heating region and a cool region. The mechanical instrument controls the back and forth motion of the liquid slug between the heating and cool regions. Additionally, the cartridge assembly includes one or more puncture elements configured to pierce the reservoirs and provide the wet reagent to the chip. The system further includes an optical detection unit with an optical light-emitting element, an optical detector, and a processing unit for analyzing the optical detection region of the chip.

### CROSS-REFERENCE TO RELATED APPLICATIONS

This application is a continuation-in-part and claims priority to U.S. patent application Ser. No. 18/032,455 filed Apr. 18, 2023, which is a 35 U.S.C. § 371 U.S. national phase entry of International Application No. PCT/US2021/055638 having an international filing date of Oct. 19, 2021, which claims the benefit of priority under Article 8 PCT of U.S. Provisional Patent Application No. 63/093,640 filed Oct. 19, 2020 and entitled “Point of Collection qPCR System.” This application is also a continuation-in-part and claims priority to U.S. patent application Ser. No. 18/032,457 filed Apr. 18, 2023, which is a 35 U.S.C. § 371 U.S. national phase entry of International Application No. PCT/US2021/055645 having an international filing date of Oct. 19, 2021, which claims the benefit of priority under Article 8 PCT of U.S. Provisional Patent Application No. 63/093,640 filed Oct. 19, 2020. This application is also a continuation-in-part and claims priority to U.S. patent application Ser. No. 18/032,459 filed Apr. 18, 2023, which is a 35 U.S.C. § 371 U.S. national phase entry of International Application No. PCT/US2021/055647 having an international filing date of Oct. 19, 2021, which claims the benefit of priority under Article 8 PCT of U.S. Provisional Patent Application No. 63/093,640 filed Oct. 19, 2020. This application is also a continuation-in-part and claims priority to U.S. patent application Ser. No. 18/032,463 filed Apr. 18, 2023, which is a 35 U.S.C. § 371 U.S. national phase entry of International Application No. PCT/US2021/055649 having an international filing date of Oct. 19, 2021, which claims the benefit of priority under Article 8 PCT of U.S. Provisional Patent Application No. 63/093,640 filed Oct. 19, 2020. The contents of the above application are all incorporated by reference as if fully set forth herein in their entireties.

### FIELD

The present invention relates to systems and related methods for the identification of nucleic acid target sequences in a sample and, in particular, relates to the identification of target amplicons using amplifications and melt curve analysis for diagnosing tuberculosis (TB) and associated antibiotic resistances.

### BACKGROUND

Melt curve analysis is a technique employed in molecular biology and genetic analysis to detect and characterize nucleic acid variations and mutations. Detecting and characterizing nucleic acid variations and mutations is of paramount importance in various scientific and clinical applications, including genotyping, disease diagnosis, genetic profiling, and monitoring of genetic variations in populations. Over the years, significant advancements have been made in nucleic acid analysis technologies, enabling researchers and clinicians to gain deeper insights into the genetic makeup of organisms and diseases.

Melt curve analysis has emerged as a powerful and widely used method for detecting sequence variations in nucleic acid samples. Melt curve analysis refers to a technique for determining a sequence variation in a nucleic acid by analyzing a melting curve of the nucleic acid. The nucleic acid can be double-stranded or single-stranded. In one aspect, a signal representing a double-stranded nucleic acid can be measured in real time. In one aspect, single-stranded nucleic acids can be analyzed, for instance, single-stranded nucleic acids can fold to form duplexes or other higher ordered structures that can then be analyzed.

Melt curve analysis also refers to a technique for determining sequence variations between two different nucleic acids by analyzing the shape of the melting curve including the melting temperature and the slope. In one aspect, melt curve analysis utilizes a fluorescent dye that intercalates with double-stranded DNA during the heating and cooling process. As the sample is subjected to a temperature gradient, variations in nucleic acid sequences are detected by the characteristic melting profiles displayed during the denaturation of the double-stranded DNA. Separately, TB, caused predominantly by the bacterium Mycobacterium tuberculosis, remains a significant global health challenge. TB affects millions of people worldwide, and its diagnosis and management require timely and accurate identification of the causative agent and any antibiotic resistances present in the infectious strains. Conventional diagnostic methods for TB, such as sputum smear microscopy and culture-based techniques, have limitations in terms of sensitivity, turnaround time, and the ability to detect antibiotic resistance quickly.

Advances in molecular biology and nucleic acid analysis have opened up new avenues for improved TB diagnosis and treatment monitoring. The emergence of nucleic acid amplification techniques, including polymerase chain reaction (PCR) and its variants, has revolutionized TB diagnostics by enabling the rapid and specific detection of Mycobacterium tuberculosis DNA in clinical samples.

### SUMMARY

According to its major aspects and briefly recited, the techniques described herein relate to a system for identifying a target amplicon for diagnosing tuberculosis and associated antibiotic resistance, including: a mechanical instrument; a cartridge assembly, including: one or more reservoirs, wherein at least one of the one or more reservoirs contains a wet reagent; a chip, including: a fluidic channel holding a liquid slug, including an optical detection region between a heating region and a cool region, wherein the mechanical instrument controls a back and forth motion of the liquid slug between the heating region and the cool region; and one or more puncture elements configured to pierce the one or more reservoirs to provide the wet reagent to the chip; and an optical detection unit, including: an optical light-emitting element; an optical detector; and a processing unit for performing analysis on the optical detection region of the chip.

In some aspects, the techniques described herein relate to a system, further including at least one heat block, wherein the chip is situated within proximity to the at least one heat block such that the at least one heat block is configured to heat at least the heating region of the fluidic channel of the chip.

In some aspects, the techniques described herein relate to a system, further including a first independent heat block and a second independent heat block, wherein the chip is situated within proximity to the first independent heat block and the second independent heat block such that the first independent heat block is configured to heat at least the heating region of the fluidic channel of the chip and the second independent heat block is configured to heat at least the cool region of the fluidic channel.

In some aspects, the techniques described herein relate to a system, wherein the optical detection region of the fluidic channel is straight.

In some aspects, the techniques described herein relate to a system, wherein the heating region of the fluidic channel is a serpentine region.

In some aspects, the techniques described herein relate to a system, wherein the cool region of the fluidic channel is a serpentine region.

In some aspects, the techniques described herein relate to a system, wherein the cool region is actively cooled or passively cooled.

In some aspects, the techniques described herein relate to a system, wherein the mechanical instrument is an overhead drum, wherein the overhead drum is positioned to depress pins onto the chip for fluidic control.

In some aspects, the techniques described herein relate to a cartridge and chip for identifying a target amplicon for diagnosing tuberculosis and associated antibiotic resistance, including: a cartridge assembly, including: one or more reservoirs, wherein at least one of the one or more reservoirs contains a wet reagent; and a chip, including: a fluidic channel, including an optical detection region between a heating region and a cool region, wherein the fluidic channel is configured to hold a liquid slug that moves back and forth between the heating region and the cool region; and one or more puncture elements configured to pierce the one or more reservoirs to provide the wet reagent to the chip.

In some aspects, the techniques described herein relate to a cartridge and chip, wherein the optical detection region of the fluidic channel is straight.

In some aspects, the techniques described herein relate to a cartridge and chip, wherein the heating region of the fluidic channel is a serpentine region.

In some aspects, the techniques described herein relate to a cartridge and chip, wherein the cool region of the fluidic channel is a serpentine region.

In some aspects, the techniques described herein relate to a cartridge and chip, wherein the cool region is actively cooled or passively cooled.

In some aspects, the techniques described herein relate to a cartridge and chip, wherein the cartridge assembly and the chip are oriented such that engaging the cartridge assembly and the chip assembly causes the one or more puncture elements to pierce the one or more reservoirs.

In some aspects, the techniques described herein relate to a cartridge and chip, wherein the chip further includes a plurality of superparamagnetic silica-coated beads.

In some aspects, the techniques described herein relate to a method of using a system for identifying a target amplicon for diagnosing tuberculosis and associated antibiotic resistance, including: providing a cartridge assembly with a chip, the chip including: a fluidic channel including an optical detection region between a heating region and a cool region of the fluidic channel, wherein the fluidic channel is configured to hold a liquid slug, and wherein the fluidic channel is configured for the liquid slug to move between the heating region and the cool region; one or more reservoirs containing a wet reagent; and one or more puncture elements configured to pierce the one or more reservoirs of the cartridge assembly to provide the wet reagent to the chip when the cartridge with the chip is assembled and inserted into a mechanical instrument; providing the mechanical instrument, the mechanical instrument including: an overhead drum, the overhead drum positioned to depress pins onto the chip for fluidic control of the liquid slug; and an optical detection unit, including: an optical light-emitting element; an optical detector; and a processing unit; inserting the cartridge with chip into the mechanical instrument; moving the liquid slug back and forth between the heating region and the cool region through the optical detection region; gradually heating the heating region; performing, via the processing unit, amplification analysis and melt curve analysis on the optical detection region using the optical light-emitting element or the optical detector.

These and other advantages will be apparent to those skilled in the art based on the following disclosure.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. In the drawings:

FIG. 1 is an illustration of an example fluidic RT-qPCR system for RT-qPCR analysis;

FIG. 2 is an illustration of an example fluidic RT-qPCR system for RT-qPCR analysis without a housing;

FIG. 3 is an illustration of an example optical detection unit;

FIG. 4A-E is an illustration of an example sample moving through an optical detection region of a fluidic channel of a chip, where the analog to digital (ADC) counts are reflective of position;

FIG. 5 is an illustration of an example chip;

FIG. 6 is an illustration of an example chart of RT-qPCR amplification time for conventional cycling;

FIG. 7 is an illustration of an example chart showing functionality according to the present disclosure for rapid RT-qPCR amplification;

FIG. 8A is an illustration of an example front cut-away view of a PCR system according to the present disclosure;

FIG. 8B is an illustration of an example first perspective cut-away view of a PCR system according to the present disclosure;

FIG. 8C is an illustration of an example left cut-away view of a PCR system according to the present disclosure;

FIG. 8D is an illustration of an example second perspective cut-away view of a PCR system according to the present disclosure;

FIG. 8E is an illustration of an example bottom cut-away view of a PCR system according to the present disclosure;

FIG. 8F is an illustration of an example third perspective cut-away view of a PCR system according to the present disclosure;

FIG. 9A is an illustration of an example top view of a cartridge/chip according to the present disclosure;

FIG. 9B is an illustration of an example perspective view of a cartridge/chip according to the present disclosure;

FIG. 9C is an illustration of an example bottom view of a cartridge/chip according to the present disclosure;

FIG. 10A is an illustration of an example chart showing fluorescence data and melt curve data for a target within a sample made possible through conventional PCR amplification systems and chips;

FIG. 10B is an illustration of an example chart showing melt curve data for a target made possible through conventional PCR amplification systems and chips;

FIG. 11A is an illustration of an example chart showing fluorescence data for an amplification stage and for a melt stage of a sample according to the present disclosure (cold block temperature data included for reference);

FIG. 11B is an illustration of an example chart showing fluorescence data for a melt stage of a sample according to the present disclosure (cold block temperature data included for reference);

FIG. 11C is an illustration of an example chart showing fluorescence data for a melt stage of a sample according to the present disclosure (cold block temperature data included for reference);

FIG. 12A is an illustration of an example chart showing fluorescence data and melt curve data for a wild-type target within a sample according to the present disclosure;

FIG. 12B is an illustration of an example chart showing fluorescence data and melt curve data for a wild-type target within a sample according to the present disclosure;

FIG. 12C is an illustration of an example chart showing melt curve data for a wild-type target within a sample according to the present disclosure; and

FIG. 13 is an illustration of a flow diagram of an example method according to the present disclosure.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Throughout this specification and the claims, the terms “comprise,” “comprises,” and “comprising” are used in a non-exclusive sense, except where the context requires otherwise. Likewise, the term “includes” and its grammatical variants are intended to be non-limiting, such that recitation of items in a list is not to the exclusion of other like items that can be substituted or added to the listed items.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

### I. Example Use Case Scenario(s)

Despite the effectiveness of melt curve analysis as an analytical tool, several challenges persist, hindering its widespread adoption and utility in certain applications. One of the significant issues is related to sensitivity and specificity. Detecting subtle sequence variations, particularly in complex genomic regions, can be challenging, leading to potential false-negative or false-positive results.

Moreover, the analysis of nucleic acid samples with high GC content or secondary structures can introduce technical complications, resulting in ambiguous or inconclusive melt curve profiles. Such challenges may limit the accurate identification of target amplicons, thereby compromising the reliability of melt curve analysis in certain experimental conditions.

As such, in one aspect, the present disclosure relates to real-time polymerase chain reaction, (qPCR), systems and methods, namely, PCR systems and methods. In particular, in one aspect, the invention relates generally to methods for analyzing a sample for the presence of one or more nucleic acids, and more particularly, to methods for conducting multi-stage nucleic acid amplification reactions, (e.g., polymerase chain reactions (PCRs)) for clinical-diagnostics and pathogen-detection, and for profiling complex samples using melt curve analysis.

For purposes of this disclosure: real-time polymerase chain reaction (real-time PCR), also known as quantitative PCR or (qPCR), is a technique that monitors amplification of a targeted DNA during real time, that can be used in quantitative analysis, rather than at the end of a polymerase chain reaction (PCR) procedure. In one aspect, qPCR is designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: 13-actin, GAPDH, 132-microglobulin, ribosomal RNA, and the like. Techniques for quantitative PCR are well-known to those of ordinary skill in the art

“Amplicon(s)” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, to a target sequence or its complement is required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, ligase chain reactions (LCRs), strand-displacement reactions (SDAs), nucleic acid sequence-based amplification (NASBAs), rolling circle amplifications, and the like. An amplicon may be single-stranded or double-stranded, or a combination thereof. An amplicon corresponds to a segment or the entire length of a nucleic acid target molecule.

“Nucleic acid amplification” or “amplification” are used interchangeably and refer to reaction in which replication of a nucleic acid sequence occurs repeatedly over time to form multiple copies of at least one segment of a template nucleic acid molecule. Amplification may generate an exponential or linear increase in the number of copies as amplification proceeds. Typical amplifications produce a greater than 1,000-fold increase in copy number and/or signal. Exemplary amplification reactions for the assays disclosed herein may include the polymerase chain reaction (PCR) or ligase chain reaction (LCR), each of which is driven by thermal cycling. Thermal cycling generally involves cycles of heating and cooling a reaction mixture to perform successive rounds of denaturation (melting), annealing, and/or extension.

Amplification may be performed with any suitable reagents. Amplification may be performed, or tested for its occurrence, in an amplification mixture, which is any composition capable of generating multiple copies of a nucleic acid target molecule, if present, in the composition. An amplification mixture may include any combination of at least one primer or primer pair, at least one probe, at least one replication enzyme (e.g., at least one polymerase, such as at least one DNA and/or RNA polymerase, and/or at least one ligase), and/or deoxynucleotide (and/or nucleotide) triphosphates (dNTPs and/or NTPs), among others.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art.

“Polynucleotide” and “oligonucleotide” are used interchangeably and each means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′-+3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context.

In clinical diagnostics and pathogen detection, the profiling of complex samples for low concentration genotypes represents a significant challenge. The rapid and accurate profiling of pathogen genotypes in complex samples remains a challenge for existing molecular detection technologies. Currently, the identification of bacterial infections relies primarily on culture-based detection and phenotypic identification processes that require several days to weeks to complete. The practical application of molecular profiling technology is limited by several factors. To replace culture, molecular approaches must capture an equally wide array of pathogens while also providing specific and sensitive identification in a turnaround time fast enough to impact clinical decision making. Moreover, the number of microbial genomes present in a clinical sample may be extremely low and/or the sample may be comprised of several different microbes. As such, current bacteria-targeted rapid screening technologies suffer from non-specific hybridization (e.g. microarrays, FISH), non-specific protein signals (e.g. protein mass spectrometry), or limited resolution of species (e.g. nucleotide mass spectrometry).

Therefore, in another aspect, the present disclosure relates to an improved integrated platform or system enabling the identification of nucleic acid sequences, such as, e.g., bacterial, viral, or fungal pathogen DNA or RNA sequences, in complex samples. In one aspects, the improved system incorporates a fluidic or microfluidic chip and instrumentation to accomplish universal nucleic acid amplification followed by melt curve analysis. The system precisely heats and unwinds post-PCR DNA amplicons in the presence of a fluorescent intercalating dye or molecular probes, for example, such that loss-of-fluorescence melt curves are generated.

In one aspect, the present disclosure provides a fluidic or microfluidic chip or cassette and associated system that integrates PCR, fluidic/microfluidic pumps or diaphragms along with heating and imaging components. The sample can include one or more target nucleic acid sequences. In another aspect, the sample can be a heterogeneous sample. In another aspect, the sample can include mammalian DNA, bacterial DNA or RNA, viral DNA or RNA, fungal DNA or RNA, or combinations thereof. In another aspect, the methods, systems, and devices of the present disclosure can perform highly sensitive, high throughput, multi-dimensional, DNA melting analysis to achieve nucleic acid sequence fingerprinting, screening, and anomaly detection with as low as single molecule sensitivity. In another aspect, the methods, systems, and devices of the present disclosure achieve simultaneous high sensitivity imaging and precisely controlled heating of small volume, low thermal mass, single molecule nucleic acid amplification reactions partitioned according to a digital assay platform.

In another aspect, a sample or reaction mixture is partitioned on a fluidic or microfluidic chip platform, including chip(s) or cassette having an array of reactions configured such that the reactions can fit into a single field of view for imaging. In another aspect, the methods may include performing automated melt curve classification with an analysis device system comprising a processor configured to perform machine learning, for example, or a neural network. The melt analysis of the amplicon may be performed by simultaneously heating and imaging the partitions to produce a melt curve for the amplicon. The method may include profiling the sequence of the target nucleic acid by comparing the melt curve for the amplicon to a plurality of melt curves from different nucleic acid sequences. The method can include automated profiling of the sequence of a target nucleic acid by use of a processor implemented computer model or algorithm that classifies the target nucleic acid based on the comparison to the reference melt curve.

“Fluidics” refers to liquids and gases, e.g., water, air, wet reagents. “Microfluidics device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, and a detection system. Microfluidics may further include valves, pumps, and specialized functional coatings on their interior walls, e.g. to prevent adsorption of sample components or reactants, facilitate reagent movement by electro osmosis, or the like. Such devices are usually fabricated in or as a solid substrate, which may be glass, plastic, or other solid polymeric materials, and typically have a planar format for ease of detecting and monitoring sample and reagent movement, especially via optical or electrochemical methods. Features of a microfluidic device usually have cross-sectional dimensions of less than a few hundred square micrometers and passages typically have capillary dimensions, e.g. having maximal cross-sectional dimensions of from about 500 μm to about 0.1 μm. Microfluidics devices typically have volume capacities in the range from 1 μL to a few nL, e.g. 10-100 nL. The present disclosure is not limited to microfluidics, and relates generally to fluidic, fluidic channels, and fluidic chips.

Therefore, in one aspect, systems and related methods according to the present disclosure include a fluidic chip or cassette, and associated system, for carrying out multiple reactions, usually amplification reactions, such as PCRs, in a single reaction chamber or in multiple reaction chambers, under fluidly closed conditions. In one aspect, a sequence of reactions is employed to amplify and detect different target polynucleotides from the same biological sample. Such reactions are usually amplification reactions, but may also include other reaction types, such as sample processing reactions, including, for example, reverse transcription reactions to convert RNA sequences into cDNA sequences, or the like.

For example, a sequential test may be conducted for TB antibiotic-resistant strains that comprises a first step of amplifying the genomic region containing a rpoB-S450L SNP (e.g., SNP in codon 450 of the rpoB gene associated with rifampin-resistant TB; SNP being a type of genetic variation where a single nucleotide differs between individuals; Rifampin being an antibiotic used in TB treatment) (“SNP450” as used herein) and a second step of performing melt curve analysis to detect an SNP450 signal.

Despite advancement in molecular methods and nucleic acid analysis assays and systems, the accurate identification of TB and its associated antibiotic-resistant varieties remains challenging. Mycobacterium tuberculosis has a complex genome, and the presence of genomic variability among different strains can complicate the detection of specific target sequences associated with TB virulence or antibiotic resistance. Simultaneously detecting multiple targets, such as drug resistance mutations and virulence markers, is crucial for comprehensive TB diagnosis. However, achieving high multiplexing capabilities with conventional PCR-based methods can be technically demanding and may lead to reduced sensitivity. Some PCR-based assays may suffer from amplification biases, where certain target amplicons are preferentially amplified over others, leading to inaccurate quantification and potential misinterpretation of results.

With the above context in mind, there is a growing need for rapid and accurate TB diagnosis, especially in resource-limited settings where TB prevalence is high, and healthcare infrastructure may be limited. The ability to simultaneously identify Mycobacterium tuberculosis and relevant antibiotic resistance markers in a single assay can significantly enhance diagnostic efficiency and patient care. In light of the aforementioned challenges and notable issues, in one aspect, the systems and methods of the present disclosure aims to provide improved identification of nucleic acid target sequences associated with TB and antibiotic resistance using amplification analysis and melt curve analysis, and aim to improve the accuracy, sensitivity, multiplexing capacity, and speed of TB diagnosis and resistance profiling, ultimately contributing to more effective TB management and control strategies.

As such, in one aspect, the systems and methods of the present disclosure provide for a low cost, point-of-care device or assay configured to quickly diagnoses (e.g., within 30 minutes) TB and associated antibiotic resistances. In one aspect, a diagnostic assay according to the present disclosure diagnoses TB within 20 minutes and determine rifampicin resistance and isoniazid resistance (i.e., a different type of antibiotic) less than 10 minutes later. In one aspect, the diagnostic assay moves a sample through a fluidic channel to reduce the effects that thermal gradients play when heating regions of the fluidic channel are not the same temperature (it also reduces the likelihood off the sample unexpectedly moving). In another aspect, a diagnostic assay according to the present disclosure consists of an instrument and a test chip. The test chip, cassette, or cartridge is configured to receive a collected sputum sample, for example. The sputum is pre-filtered and processed, then dispensed into an input port of the cartridge, for example. Next, the input port is closed, and the cartridge is inserted into the mouth of a related diagnostic system. The instrument detects that there is a chip and runs the protocol as described herein.

In one aspect, an extraction buffer in the input port consisting of GITC and ethanol, for example, lyses the cells in the sample and exposes their genetic material. The cam inside the instrument turns to press the pins that actuate a flexible membrane or diaphragm on top of the chip, driving the liquid through the fluidic channels of the chip, cartridge, or cassette. This sequence first moves the extracted sample liquid through a channel in which there is a spot of dried beads (e.g., magnetic hydrophobic beads, superparamagnetic silica-coated beads). The liquid slowly moves back and forth across the dried beads to suspend them in solution. Any DNA exposed during cell lysis then sticks to the magnetic beads. The liquid slug is moved further through the first channel, through a burst valve, and into a larger main channel. A magnet in the instrument holds the beads in position, and the liquid is pushed into the waste port. Moreover, liquid from a well of wash buffer is pushed into the main channel, displacing any remaining extraction liquid and washing the beads. The waste liquid is then pushed into the waste port. Moreover, liquid from a second well of wash buffer is pushed into the main channel, ensuring that the beads are free of GITC, ethanol, or anything else that might inhibit PCR. An elution buffer from a well is then driven into the main channel, releasing the purified DNA from the magnetic beads. This eluate is driven through the main channel, through a burst valve, into five parallel “PCR channels” containing dried master mix, primers, and probes. These components are rehydrated by mixing the liquid across the dried spots. Then, the liquid is pushed to the hot block, where it is held for 90 seconds to activate the DNA polymerase. Starting on the hot block, the liquid is driven back and forth from hot block to cold block, across an optical detection region where a camera sensor, for example, measures and logs the fluorescence of each liquid slug. The liquid pauses for about 0 seconds to about 1 minute in the heating region and for about 0 seconds to about 1 minutes on the heating/cool region during each of the up to 50 amplification cycles (or more). In another example, the pause may be for about 3 seconds to about 8 seconds in the heating region and for about 3 seconds to about 8 seconds on the heating/cool region during each of the amplification cycles. Once PCR is complete, the hot block is cooled to 65 C, the cold block is set to 65 C, and the liquid is pushed to the hot block. Then, both heaters slowly ramp up their temperatures by 0.X degrees per second until they reach 95 C (e.g., by about 1-degree C. per second, or by about 0.5 degrees C. per second). The liquid slugs are pushed from one block, across the optical detection region, to the other block once every few seconds (depending on the protocol). The photodiode (or, for example, a light detector, camera, optical measurement device, individually or in combination) takes fluorescence measurements of the liquid slugs every time they pass through the optical detection region as happens during the real-time PCR sequence. When the heaters reach 95 C, the run is complete and the instrument processes the data. If amplification of the target TB sequence occurs then the sample is “positive,” and if not, then “negative.”

In one aspect, melt curve data is analyzed or compared to determine whether certain SNPs are present in the sample, and therefore whether the detected strain of TB is resistant to certain antibiotics. In another aspect, fluorescence data is analyzed or compared to determine whether certain SNPs are present in the sample, and therefore whether the detected strain of TB is resistant to certain antibiotics. In another aspect, amplification data and melt curve data is analyzed to determine whether certain SNPs are present in the sample, and therefore whether the detected strain of TB is resistant to certain antibiotics.

### II. Systems and Methods

Aspects of the present disclosure are directed to fluidic chips or cassettes, and associated system, for carrying out multiple reactions, usually amplification reactions, such as PCRs, in a single reaction chamber or in multiple reaction chambers, under fluidly closed conditions.

In one aspect, a system for detecting and making real-time adjustments to positional control of a fluidic volume moving through an optical detection region in a fluidic channel is disclosed. The system includes a mechanical instrument configured with a chip comprising a fluidic channel, wherein the mechanical instrument controls the motion of the fluidic volume. The chip is further comprised of an optical detection region configured for use with an optical detection unit(s) or device(s). The system is further equipped with one or more independent heat blocks. The chip is positioned at least partially on the one or more independent heat blocks so that the fluidic channel is within close proximity to the one or more independent heat blocks. Further, the system is equipped with an optical detection unit that comprises an optical light-emitting element, and two or more light emitting diodes with a dual band filter. The optical detection unit of the system further comprises an optical detector, which may be a plurality of detection diodes (or cameras, for example), and a processing unit for performing analysis on the optical detection region of the chip. In one aspect, the chip itself is moved to and from an optical detection region within the instrument.

In another aspect, a method for detecting and making real-time adjustments to positional control of a fluidic volume moving through an optical detection region in a fluidic channel is disclosed. The method comprising preparing a sample with a fluorescent marker. Next, configuring a chip to receive the sample, wherein the chip has a fluidic channel for the sample to flow along, and an optical detection region that allows for optical light transmission to the fluidic channel. Next, applying the sample to the chip and applying the chip to two heating arrays. Then, initiating an optical detection unit, wherein initiating the optical detection unit is configured to illuminate one or more LED's and to activate an optical detection diode. Next, initiating a mechanical instrument to depress regions of the chip, wherein the depressed regions cause the fluid within the chip, including the sample, to move along the fluidic channel and across the two heating arrays. Then detecting, by the optical detection unit, presence of the sample and signal output of the sample within the optical detection region. The method then adjusting a motion control script based on at least the signal output, wherein the motion control script adjusts at least a start and/or stop of the mechanical instrument.

In another aspect, a sequence of reactions is employed to amplify and detect different target polynucleotides from the same biological sample. Such reactions are usually amplification reactions, but may also include other reaction types, such as sample processing reactions, including, for example, reverse transcription reactions to convert RNA sequences into cDNA sequences, or the like. In another aspect, such methods are carried out in a fluidly closed reaction system by the following steps: (i) mixing a portion of a sample with amplification reagents to form a reaction mixture; (ii) amplifying in the reaction chamber one or more target polynucleotides to form one or more amplicons in the reaction mixture; (iii) detecting the one or more amplicons to determine the presence or absence of the one or more target polynucleotides in the sample; (iv) removing the reaction mixture from the reaction chamber after detection; and (v) repeating steps (i) through (iv) until the presence or absence of the plurality of target polynucleotides is determined. In the step of mixing, the sample portion and the amplification reagents may be combined and mixed in a cavity or reagent reservoir after which they are transferred to the reaction chamber for amplification. Alternatively, the sample portion and the amplification reagents may be combined and mixed in the reaction chamber directly.

Aspects of the present disclosure also are directed to system for conducting an assay. Example systems include a cartridge assembly and a chip assembly. The cartridge assembly can include a first surface having a first seal, a second surface having a second seal, and one or more cavities or reservoirs positioned between the first surface and the second surface. The chip assembly can include: a fluidic channel, and one or more puncture elements configured to pierce the second seal to provide the wet reagent to the chip assembly. In another aspect, the cartridge assembly or chip assembly can also include: a fluidic channel, a cavity or reservoir, and one or more puncture elements configured to pierce a seal to fluidly link (e.g., to make the fluid channel in fluid communication with) the cavity or reservoir. The puncture elements are themselves in fluid communication with the fluidic channel prior to a puncture step. In another aspect, a puncture step can be associated with assembly of the cartridge or chip, or with use of the cartridge or chip.

In another aspect, the present disclosure is directed to a method including providing a biological sample to a cartridge assembly, engaging the cartridge assembly with a chip assembly to transfer the biological sample to the chip assembly, moving the biological sample through the fluidic channel, and exposing the biological sample to a temperature.

Aspects of the one or more puncture elements can include a hollow structure. For instance, in some implementations, the puncture elements can have a needle structure, the needle structure allowing fluid from the reservoir to flow though the hollow interior of the needle to reach the fluidic channel.

In one aspect, the cartridge assembly and the chip assembly may be oriented so that engaging the cartridge assembly and the chip assembly causes the one or more puncture elements to pierce the second seal and the compressible layer to contact the chip assembly to fluidically seal the fluidic channel. For example, the cartridge assembly and the chip assembly may include aligning feature(s) so that the cartridge assembly and the chip assembly are oriented so that the puncture elements on the chip assembly are aligned with the reservoirs. In this manner, upon engaging the cartridge assembly and the chip assembly, the puncture elements pierce the second seal at the reservoirs to provide the wet reagent to the chip assembly.

Additionally, in some implementations, engaging the cartridge assembly and the chip assembly can compress the assemblies together to fluidically seal the fluidic channel. As an example for illustration, the compressible layer may be configured to deform upon engaging the cartridge assembly and the chip assembly to produce an air-tight or substantially air-tight seal. More particularly, the cartridge assembly and the chip assembly can be oriented so that engaging the cartridge assembly and the chip assembly causes the one or more puncture elements to pierce the second seal and the compressible layer to contact the chip assembly to fluidically seal a top of the fluidic channel.

### III. With Reference to the Figures

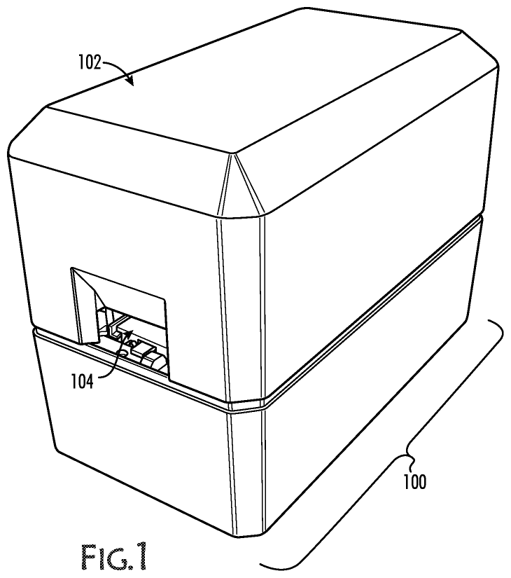

Turning now to the figures, FIG. 1 is an illustration of an example fluidic RT-qPCR system 100 having a housing 102. The housing 102 protects the internal elements, including the mechanical instrument for pushing pins into the membrane of a chip through a cartridge, as well as the processing equipment. The cartridge slides into the cartridge insert 104. The chip is configured within the cartridge, where a sample and/or fluid is first placed on the chip, and then the chip is inserted into the cartridge insert 104. Dimensions for the system range in size (e.g., 140 mm×140 mm×170 mm), allowing for the system to fit within an existing laboratory environment b

As discussed, the qPCR system 100 is a real-time qPCR system (RT-qPCR), wherein the sample is observed in real-time as the amplification procedure is conducted. The fluidic RT-qPCR system 100 is designed to handle many of the tasks which may have traditionally been performed by a technician. The cartridge with chip assembly is consumable, allowing single use and disposal.

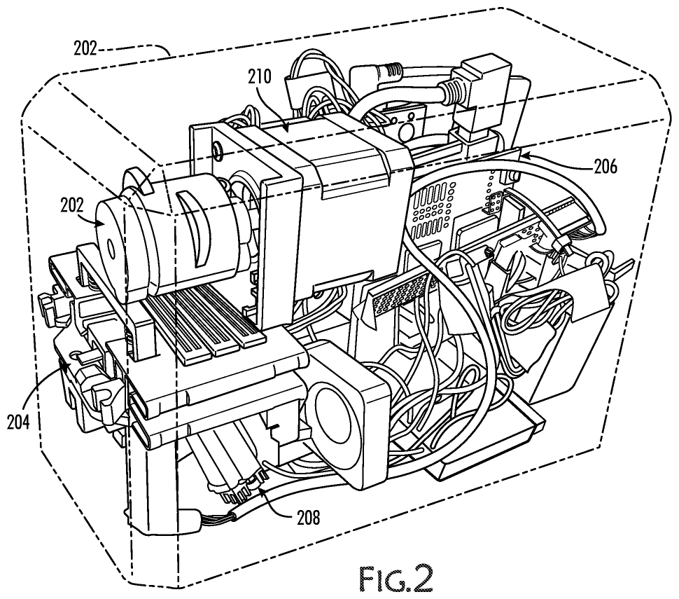

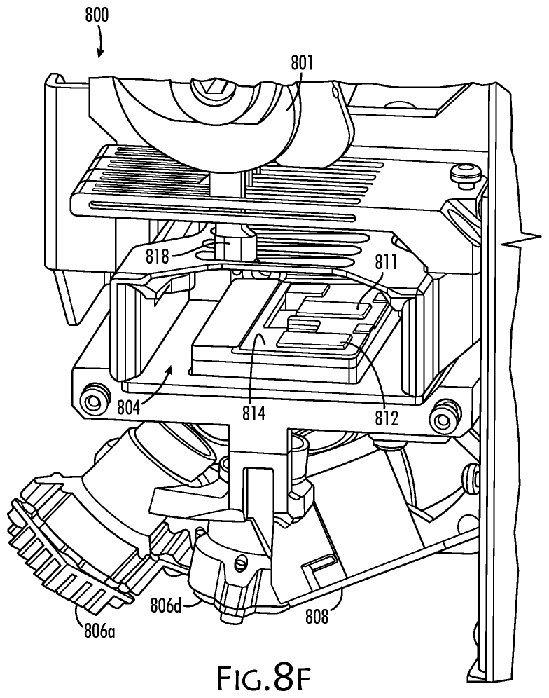

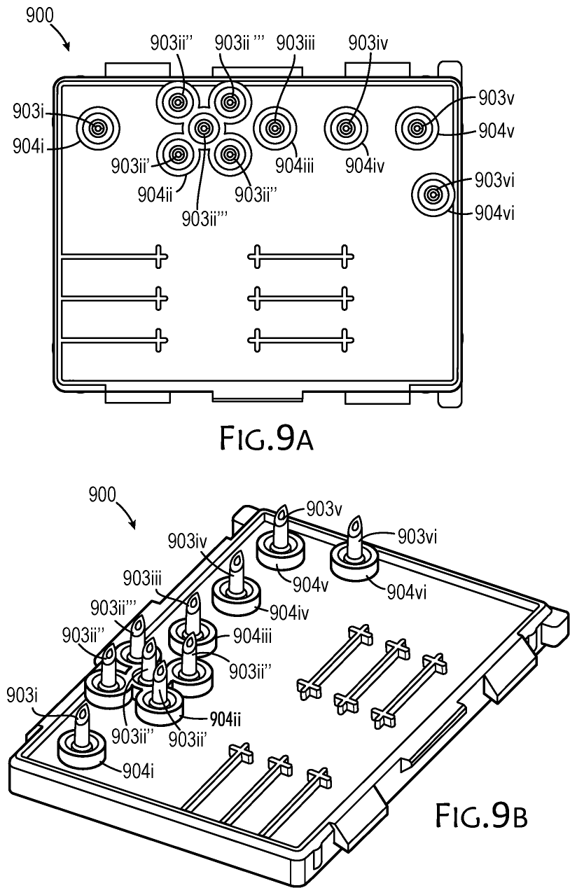

Referring now to FIG. 2, an illustration of an example fluidic RT-qPCR system 200 for RT-qPCR analysis without a housing 204 is shown. In the example, a mechanical instrument 201 orbits around a central axis. The mechanical instrument 201 is equipped with nodes or lobes, wherein orbiting the nodes or lobes impact a set of pins, the pins press onto a rubber membrane of a chip, thus allowing fluid trapped within the chip to be cycled to different zones. The differing zones include heating regions, cool regions, magnet regions, and/or capacitive array regions. A further elaboration of the various zones of an example chip are disclosed in FIGS. 5 and 9. Moreover, the mechanical instrument 201 may be an overhead drum, a drum, or a cam that is rigid in nature with a smooth exterior and nodules for depressing pins onto the surface membrane of a chip. The overhead drum or cam may be comprised of a metal or hard polymer; further, a motor 210 configured to the mechanical instrument allows for start and stop, as well as changing the acceleration and velocity of the drum. The motor 201 connects through a shaft or a gearbox to drive the overhead drum or cam. Input received from the optical detection unit, and/or the capacitance array, allows for configuration of a control algorithm for the mechanical instrument 201.

In one aspect, an algorithm is defined within the logic of the processing unit, wherein a certain measure of analog to digital (ADC) counts on a fluorescent marker, for example, may send a feedback loop to either stop the mechanical instrument after a specified time, such as ten seconds, for example, from passing the optical detection region. Similarly, the mechanical instrument may be triggered to begin rotation around the central axis when ADC counts have cleared an optical detection region and have spent a specified period of time on a heating unit.

A consumable cartridge is configured to be received by the cartridge insert 204 such that the cartridge is aligned with the various pins oriented beneath the mechanical instrument 201. As the motor 210 engages, in response to either the processing unit or system pre-defined function, or as part of the initial system parameters, the mechanical instrument 201 turns around a central axis, the lobes press the pins to cycle fluid within the fluidic channel of the cartridge or chip.

The processing unit may be equipped on a single printed circuit board and may have multiple components such as a GPU, RAM, SSD, along with peripherals and I/O for additional input and output to peripheral devices. It is contemplated that the system herein may communicate over a data cable or through wireless protocols, and may be adapted and configured to a cloud environment where a cluster of devices may form a system that can perform diagnostics and testing on a plurality of devices.

In one example, the sample may be prepared with a fluorescent marker (dye), such as that of FAM™, HEX™, ROX™, TET™, JOE™, VIC™, NED™, PET™, TAMRA™, or any other fluorescent dye utilized in DNA sequencing. Many of these dyes are excited at a single wavelength of 488 nm, but emit at distinctly different wavelengths. The table below provides examples of various fluorescent dyes that may be incorporated within the disclosure herein.

TABLE 1
Example of Fluorescent Dye

| Name | λₘₐₓ/nm (absorption) | λₘₐₓ/nm (emission) |
|---|---|---|
| FAM ™ | 494 | 518 |
| HEX ™ | 535 | 556 |
| ROX ™ | 575 | 602 |
| TAMRA ™ | 555 | 580 |
| JOE ™ | 520 | 548 |

In another example, the sample may be prepared with magnetic beads, or beads for preparation of a DNA sample, such as those manufactured by ACRO Biosystems™ for binding to nucleic acid compounds. There are a multitude of manufacturers of magnetic beads, and the beads may be selected for properties that fit the specific sample to be analyzed. In yet another example, the sample may have both the fluorescent marker/dye and magnetic beads applied for further processing and utilization of the optical detection system and the capacitive and magnetic arrays as further disclosed herein.

System parameters for a processing unit 206 that drives the system, including the mechanical instrument 201, may be the start and stop time, the length of a cycle, where a cycle is the amount of time the mechanical instrument is in motion, the acceleration of the mechanical instrument, the velocity of the system instrument, as well as many other settings such as the temperatures of the various heating regions (e.g., temperature of the resistive heating element(s)), the power to the capacitive regions, the setting for the optical detection unit (e.g., detection signals or zero detection signals), as well as other parameters as disclosed herein.

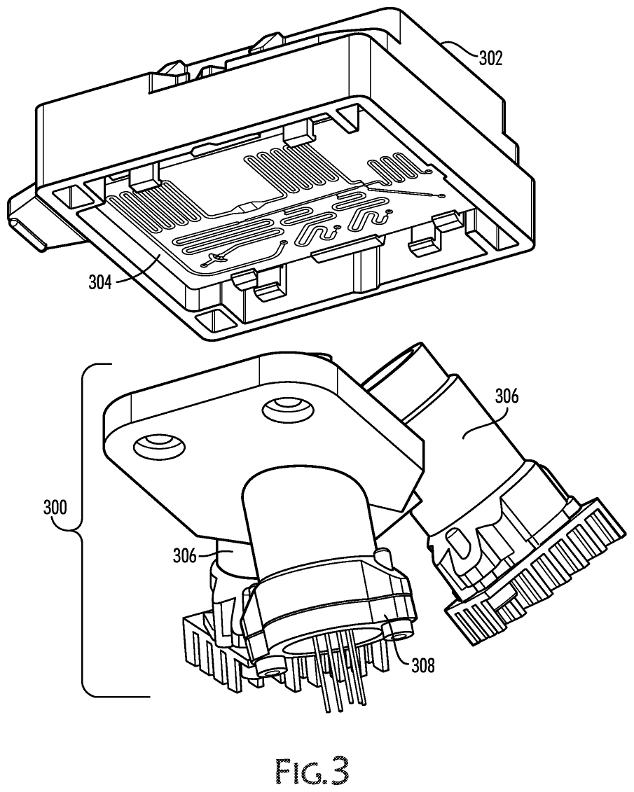

Referring now to FIG. 3, an illustration of an example optical detection unit 300 is shown. An optical detection unit 300 is configured below the cartridge 302, which houses the chip 304, wherein the prepared sample is moved. Depicted on the chip 304 is a fluidic channel in which the mechanical instrument, through interaction with pins on the rubber membrane on the surface of the chip 304 will move the sample and other fluids to specific regions or zones within the chip 304. Not depicted in FIG. 3 are the heating regions or heating zones, along with respective heating units, the magnetic zone and the capacitive arrays and zones. These elements are positioned on the bottom of the chip 304 and correspond to the various functionality disclosed herein. For example, the serpentine pathway on the left and right side of the chip, in one example, may be situated over two separate heating elements or over one heating element and one cooling element. One element comprising a hot region or zone (95-98 C) on the fluidic channel and a cool region (55-60 C) on the fluidic channel. The hot region forming a first heat block or unit, and the cool region forming a second heat block or unit. This type of heating arrangement is utilized for the amplification process. Between the two heating regions is an optical detection region, wherein the optical detection unit is focused for observation of the fluorescent dye within the sample.

The optical detection unit 300, is configured with an optical light-emitting element such as diode 306, an optical detector such as a detection diode 308, and a processing unit for performing analysis on the optical detection region of the chip 304. The processing unit may further be configured with an onboard timer, including a system timer for the processor, such timer may be used in determining peripheral device interaction. The optical light-emitting diodes 306 emit at an excitation value for the particular dye or marker within the sample to produce results detectable by the detection diode 308. As a sample moves, by force from the mechanical instrument pushing pins onto the rubber membrane of the chip, the fluid crosses the optical detection region wherein the optical detection unit 300 performs analysis on the sample. The optical detection region is an area where the optical detection unit may perform as intended. This type of detection is often referred to as dynamic detection, as the optical detection unit 300 is performing detection as the fluid cycles, and/or as amplification is occurring in real-time. Therefore, the motor 310 is capable of adjustments of on and off, or a change in acceleration or velocity, while the fluid is being cycled. Therefore, in one aspect, the optical detection unit 300 can serve as a control for the motor 310 as well as a verification and detection system. Example light emitting diodes 306, include those manufactured by Lumileds™ such as LED lighting color series Blue 470 nm for dyes or markers with the specific wavelength. Typical current max is 1 amp with a luminous flux of 35 lm and a viewing angle of 125 degrees.

In another aspect, the optical detection unit 300 forms an assembly that may have computational power built within it, or may connect through data cables or wirelessly to a processing unit housed elsewhere on the system. Further, microcontrollers and motors may be applied to the optical detection unit 300 for fine tuning diode angle(s), or adjusting the chip 302. Such improvements in moving a unit or assembly will be known to those of skill in the art, that certain refinement in optical imaging requires precision with location of diodes and light emitting sources. Further, any number of diodes may be applied, in this example three optical light emitting diodes are disclosed. However, in other embodiments one, two, or more may be used. Similarly, with the detection diode 308, in this example two detection diodes are disclosed, however, the number of detection diodes will vary with the system goals.

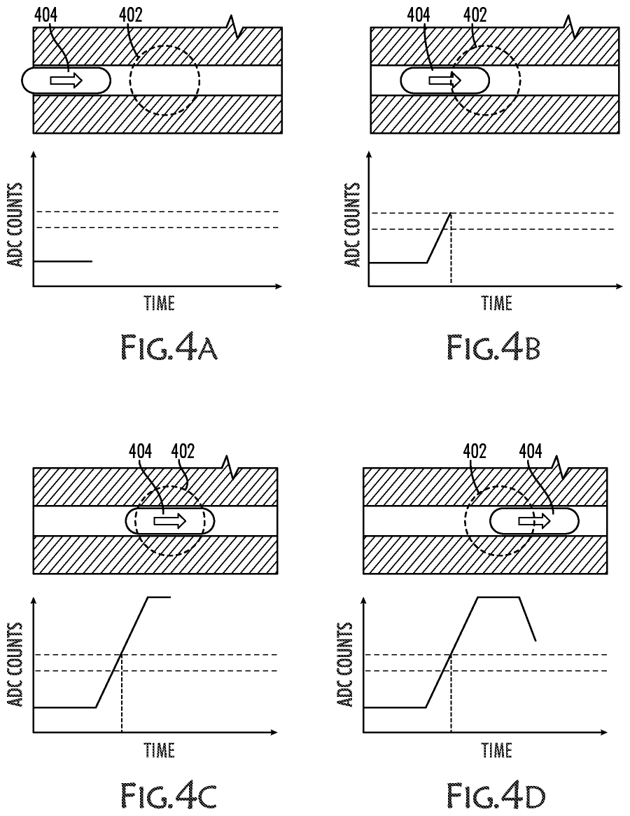

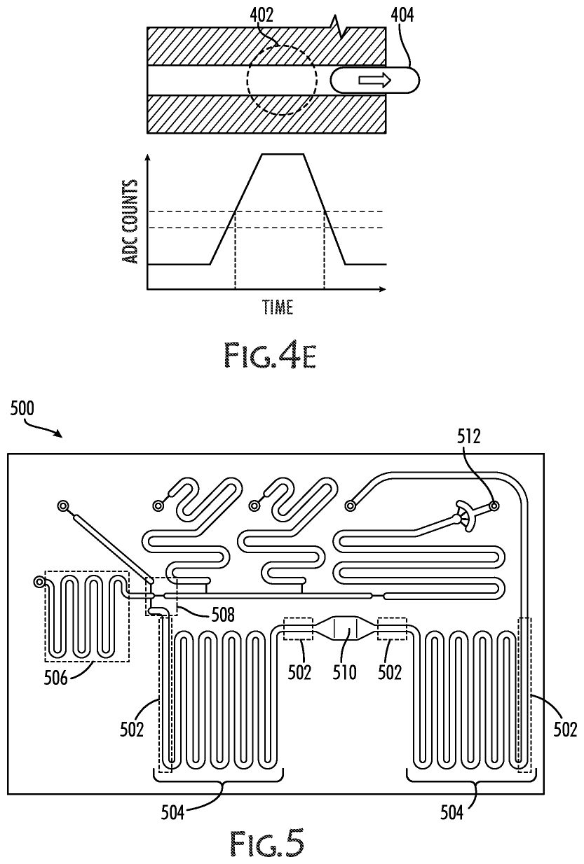

Referring now to FIGS. 4A-E, illustrations of an example sample moving through an optical detection region of a fluidic channel of a chip are shown. In the example, the analog to digital (ADC) counts are reflective of the position of the sample. In FIG. 4A, the sample 404 is moving through the fluidic channel of the chip, and is shown moving from one heating region to another heating region by the mechanical force on the membrane of the chip causing air within the chip to move the sample through the fluidic channel. The sample 404 is moving towards the detection region 402, where a voxel is located for better access for the optical detection unit. A light emitting diode is placed to direct light up to the bottom side of the chip where the sample 404 is moving. As the sample 404 has not reached the optical detection region 402, the ADC counts remain at a baseline.

In FIG. 4B, the sample 404 begins entering the optical detection region 402, and ADC counts begin to increase. This increase is due to the diode reflecting off the fluorescent dye or marker, and the diode receiving more counts. In FIG. 4C, the sample 404 is centered on the optical detection region 402, and ADC counts peak. This reading informs the system the sample is within the voxel or that a majority of the sample is moving towards the next heating region. The motor 410 may be controlled to run until counts drop, or to pause for additional readings at the optical detection region 402. In FIG. 4D, the sample 404 is moving out of the optical detection region, indicating that the sample is moving towards the heating element and away from the voxel. At this stage ADC counts begin to drop, and a distinct sinusoid wave or curve is formed to indicate sample movement. This information is then utilized to inform a technician as well as the motor 410 on relative positioning of the sample, the status of the system, and the amount of cycles performed.

In FIG. 4E, the sample 404 has egressed through the optical detection region 402 and is entering a heating region, or is within a capacitive array region, or is progressing along the fluidic channel as prescribed. If the sample 404 is not detected with a similar curve the system may alert a technician or throw an error that the sample 404 is not being cycled, or that the run needs to start again. The curve forming from the amount of counts over time, indicating movement of the sample.

Referring now to FIG. 5, an illustration of an example chip 500 having a fluidic channel(s) 502. The fluidic channel(s) 502 navigate throughout the chip 500. Additional chip designs and ornamentation are contemplated, so long as the principals disclosed herein remain, and such additional configurations form a part of this disclosure. On the top side of the chip 500 is a rubber or latex membrane, which may consist of rubber or other material that allows for stretching when contacted by pins being driven by a motor attached to a mechanical instrument, such as a mechanical drum or cam shown in FIG. 2. The chip may also comprise several other layers, such as a capacitive layer, and layers for insertion of probes or other diagnostics.

In FIG. 5, the sample start 512 is where the sample first enters the chip 500 from the more general cartridge and begins the process. The mechanical instrument moves the sample through the sequence of channels to a fluid hub 508, such as a magnetic region 506, which is designed when the sample is prepared with magnetic beads to hold portions of the sample bound to said beads within the magnetic region 506.

In FIG. 5, two heating regions are disclosed, one a hot region and the other a cold region, with an optical detection region 510 therebetween. Temperatures are defined for optimal amplification of nucleic acids or for the processes and methods described herein. The hot region (95-98 C) and a cool region (55-60 C) may be heated to the specific temperatures by two thermal heating units. In other examples one heating element with variability of heating zones may be used, or in additional examples more than two heating elements may be used.

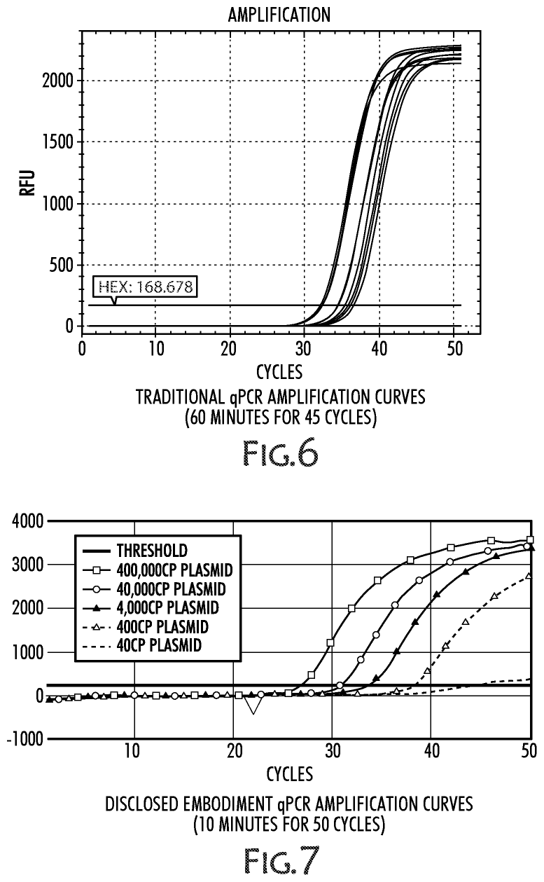

FIG. 6 is an illustration of an example chart of RT-qPCR amplification time for conventional cycling. Amplification of the target within the sample is disclosed along with an average timeline of cycles. In particular, in FIG. 9, a conventional RT-qPCR system is shown as accomplishing 45 amplification cycles and 60 minutes.

FIG. 7 is an illustration of an example chart showing the functionality disclosed herein for rapid RT-qPCR amplification. The system benefits from the feedback loop established from the optical detection unit, and the capacitive array, in coordination with the motor 1010 and mechanical instrument. Therefore, the disclosures herein further provide systems and methods for detecting and making real-time adjustments to positional control of a fluidic volume, such as a sample, moving through an optical detection region and/or a capacitive region in a fluidic channel of a chip. In particular, in FIG. 10, a RT-qPCR system according to the present disclosure is shown as accomplishing 50 amplification cycles in 10 minutes.

In one aspect, real-time adjustments are made through coordination with the optical detection unit, and the capacitive array sensor, allowing for coordinated feedback and an algorithm to control motor function of the mechanical instrument. This arrangement results in reliable, and fast RT-qPCR amplification and melt curve analysis, in a self-contained, automated system that can be operated by an unskilled user with minimal training.

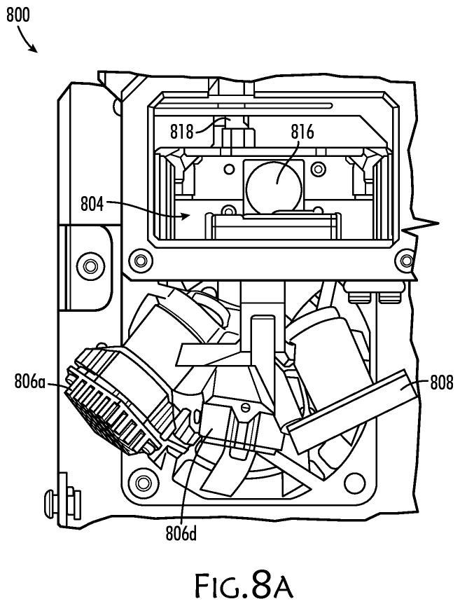

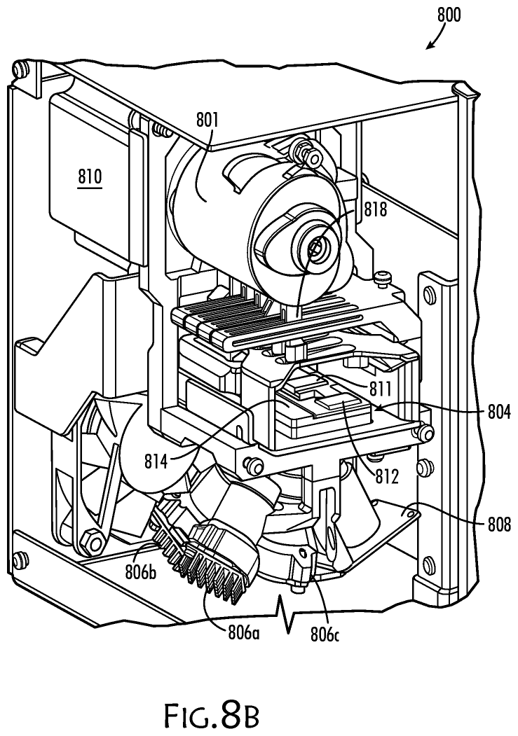

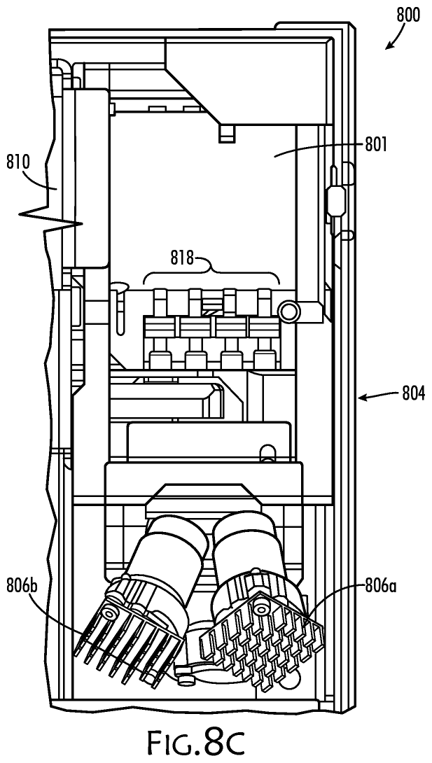

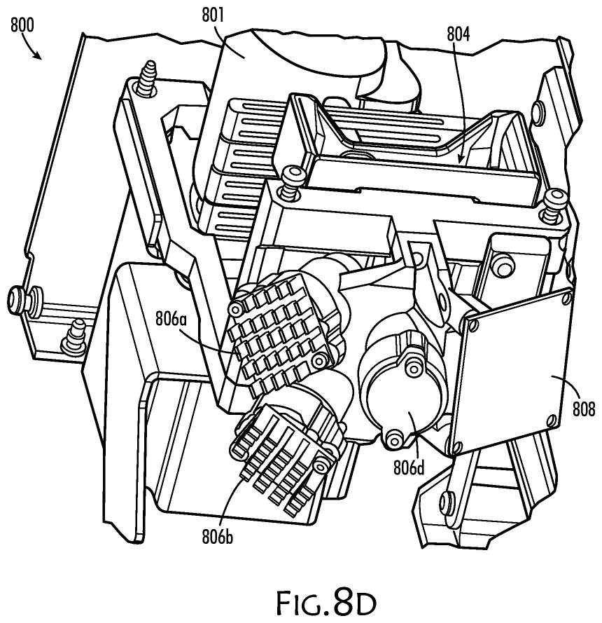

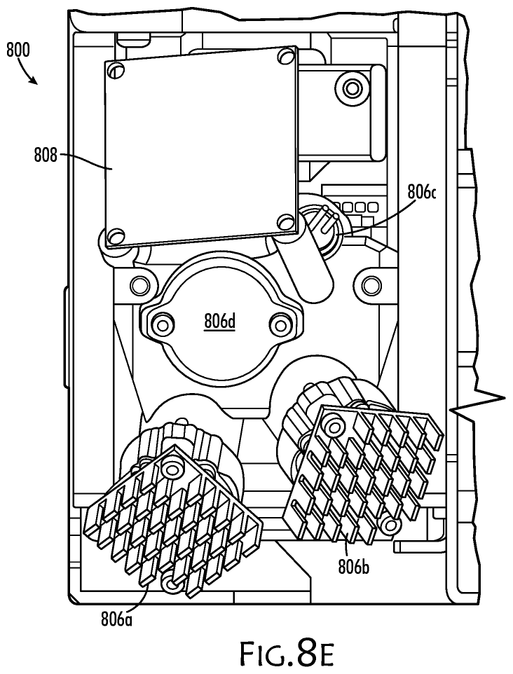

Referring now to FIGS. 8A-8F, an illustration of an example PCR system according to the present disclosure is shown. In particular, a front cut-away view (through the housing) of the PCR system 800 is shown in FIG. 8A; a first perspective cut-away view of the PCR system 800 is shown in FIG. 8B; a left side cut-away view of the PCR system 800 in shown in FIG. 8C; a second perspective cut-away view of the PCR system 800 is shown in FIG. 8D; a bottom cut-away view of the PCR system 800 in shown in FIG. 8E; and a third perspective cut-away view of the PCR system 800 is shown in FIG. 8F. In some aspects, the PCR system 800 includes an example of an optical detection unit comprising a blue LED diode 806a, an amber LED 806b (best seen in FIG. 8B), and a white LED 806c (best seen in FIG. 8B), and a photodiode 806d as well as an HD Resolution and Low Light camera sensor 808 (e.g., an embodiment of an optical detector of a detection unit) with, for example, a 2 megapixel IMX323 Color COMS sensor configured to capture minimum illumination of 0.01 Lux@F1.2.

In some aspects, the PCR system 800 also includes an annealing/extension/melt heat block 811 (best seen in FIG. 8B) and a denaturation heat block 812 situated within the cartridge insert 804 (best seen in FIG. 8B), as well as a capacitive sensor 814 (best seen in FIG. 8B). In one aspect, the annealing/extension/melt heat block 811 and the denaturation heat block 812 may be situated above the capacitive sensor 814 within the cartridge insert 804, and the diodes 806 and HD Resolution and Low Light camera sensor 808 may be situated below the cartridge insert 804 area. Moreover, the PCR system 800 includes a chip presence sensor 816 as well as membrane driving pins 818 (both within the cartridge insert 804).

A mechanical instrument 801 configured as a cam or drum orbits around a central axis. The mechanical instrument 801 is equipped with nodes or lobes, whereby orbiting the nodes or lobes impacts the membrane driving pins 818 such that the membrane driving pins 818 press onto a rubber membrane of a cartridge/chip for the system 800 within the cartridge insert 804. A motor 810 (best seen in FIG. 8B) allows for start and stop, as well as changing the acceleration and velocity of the drum. The motor 810 connects through a shaft or a gearbox to drive the overhead drum or cam 801. Input received from, for example, the diode(s) 806 as well as the HD Resolution and Low Light camera sensor 808 may facilitate control and operation of the motor 810.

Referring now to FIGS. 9A-9C, an illustration is of an example cartridge/chip 900 having puncture elements 903i-903vi surrounded by sealing features 904i-904vi, and fluidic channels 901a-901e fluidically connected to the puncture element 902ii, according to the present disclosure is shown. In particular, a top view of the chip 900 is shown in FIG. 9A; a perspective view of the chip 900 is shown in FIG. 9B; and a bottom view of the chip 900 with an example fluidic channel design is shown in FIG. 9B. The chip 300 in FIG. 9C also includes at least one filter 904, at least one burst valve 908 and channel constriction 918.

More specifically, the fluidic channels 901a-901e are fluidically connected to the puncture elements 902ii (in particular, the fluidic channel 901a is connected to puncture element 903ii; the fluidic channel 901b is connected to puncture element 903ii″; the fluidic channel 901c is connected to puncture element 903ii′″; the fluidic channel 901d is connected to puncture element 903ii″″; and the fluidic channel 901e is connected to puncture element 903ii′″″) such that fluid from a reservoir, for example, may be transferred from the reservoir to the fluidic channels 901. For instance, the puncture elements 903i-903vi may be hollow so that fluid in the reservoir travels through the puncture elements 903i-903vi to a region of the fluidic channel 901a-901e, respectively, below the puncture elements 903i-903vi. Moreover, each of the fluidic channels 901a-901e include serpentine regions 906, 910, 914, 916, respective points of entry 902ii′-902ii′″″ for fluids contained in the reservoirs of the chip 900, and optical detections regions 912.

In this way, in one aspect, the chip 900 is configured to separate a sample into the five independent fluidic channels 901a-901e such that each channel can contain a different reaction with different reagents. This allows for multiplexing up to 10 different genetic targets (five fluidic channels and two optical channels), and allows for loss-of-florescence melt curve analysis or any other type of melt curve analysis on each independent fluidic channel 901a-901e (via the optical detection regions 912 of each fluidic channel 901a-901e). Moreover, in one aspect, the chip 900 may allow for multiplexing up to 15 different genetic targets (five fluidic channels and three optical channels). In another aspect, the chip 900 may have two sets of five fluidic channels (or three sets, etc.) and a corresponding increasing number of optical channels (4, or 5, etc.). In another aspect, the chip 900 may have 8 to 10 fluidic channels (one, two, or three sets, for example). In another aspect, when the chip 900 is inserted into the system 800 of FIGS. 8A-8F, for example, the chip is orientated such that the serpentine region 916 (which is downstream of the serpentine region 914) lines up with and is above the heat block 812, and such that the serpentine region 914 lines up with and is above the heat block 811 (or a cool block, e.g., an active cold block such as an air cooled region or a hydraulically cooled region, or a passive cold block such as a region in the absence of any elements transferring heat), and such that the optical detection region 912 is above and in line with the viewing angle of the optical detection unit.

FIGS. 10A-10B is an illustration of two example charts showing fluorescence data and melt curve data made possible through conventional PCR amplification systems and chips. Fluorescence data and melt curve data for a target within a sample from a conventional system is disclosed in FIG. 10A, and melt curve data for the target from the conventional system is disclosed in FIG. 10B.

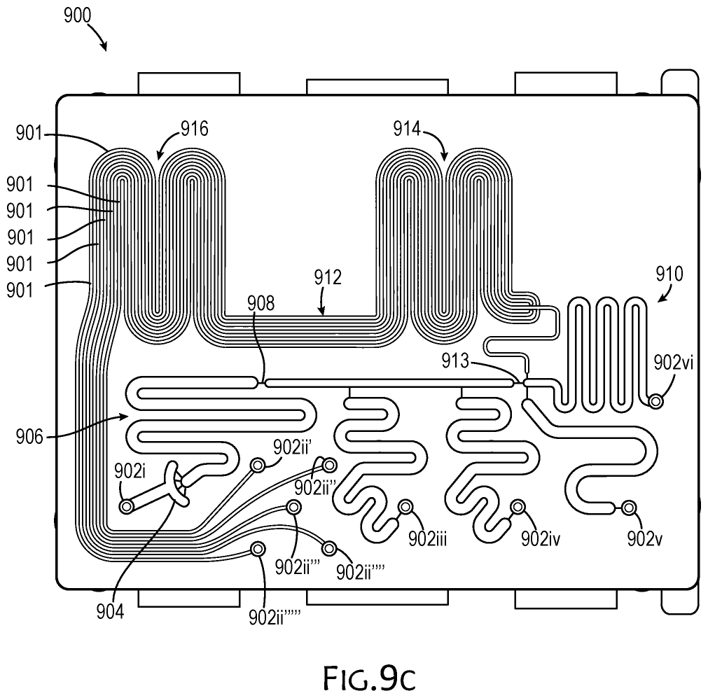

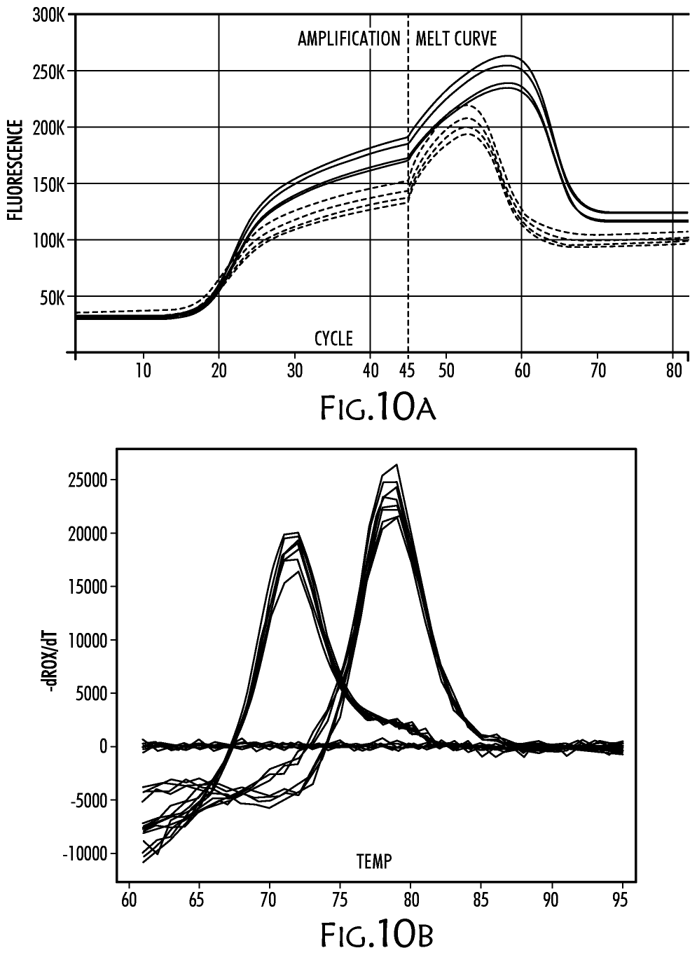

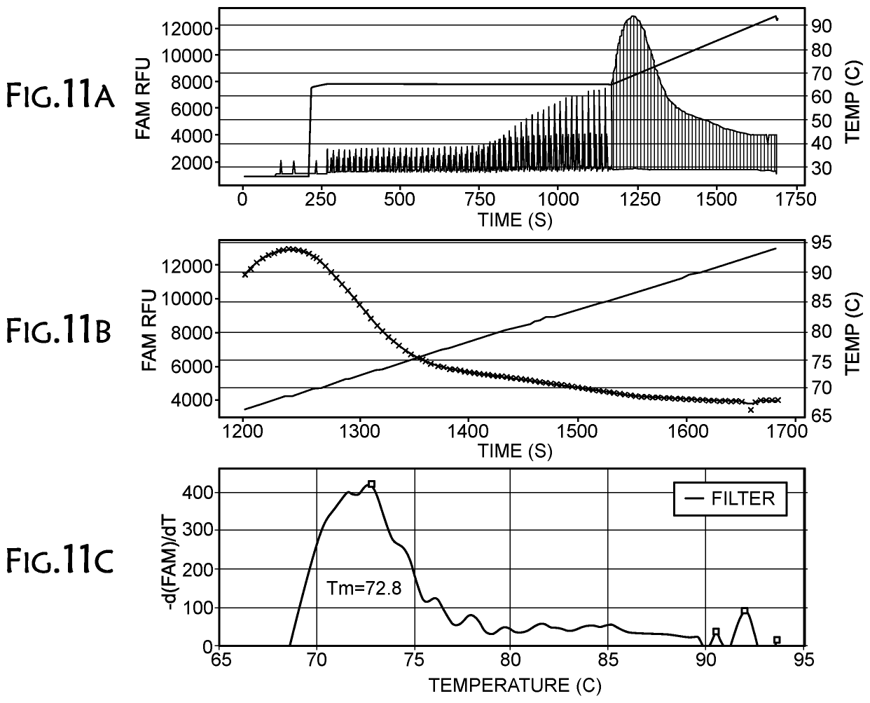

FIGS. 11A-11C is an illustration of three example charts showing fluorescence data for an amplification stage and/or for a melt stage of a sample made possible through PCR amplification systems and chips according to the present disclosure. Fluorescence data for an amplification stage and for a melt stage of a sample according to the present disclosure is disclosed in FIG. 11A; fluorescence data for a melt stage of a sample according to the present disclosure is disclosed in FIG. 11B; and fluorescence data for a melt stage of a sample according to the present disclosure is disclosed in FIG. 11C. In particular, FIG. 11C shows that the system and chip according to the present disclosure can provide targeted amplification and detection of a specific target SNP 450 signal within a sample. Cold block temperature data is included in FIGS. 11A-11C for reference.

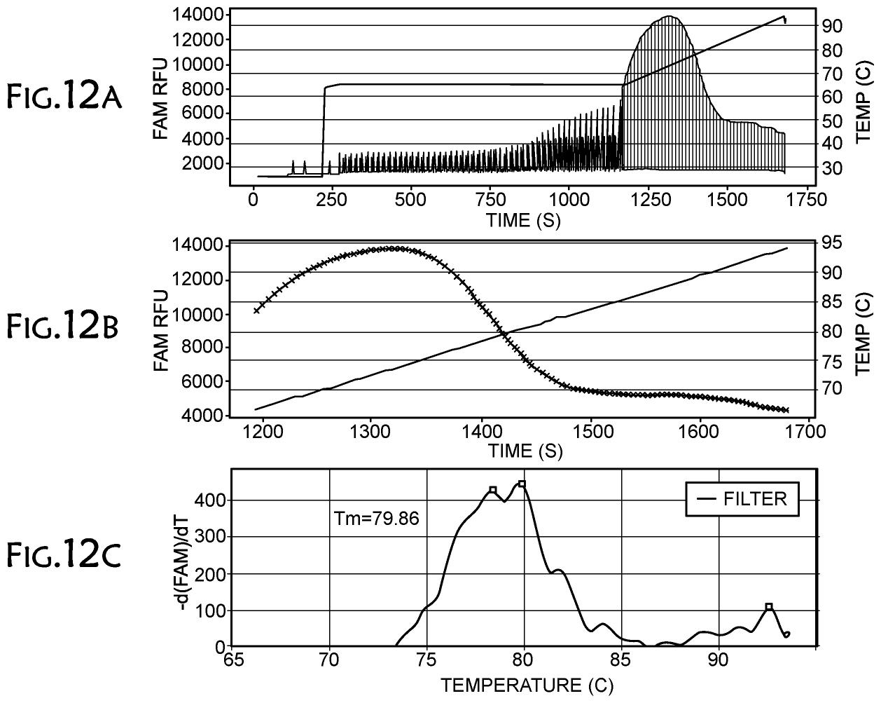

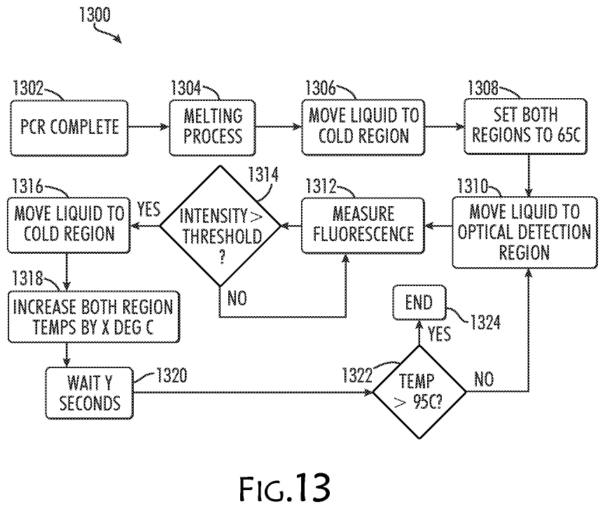

FIGS. 12A-12C is an illustration of three example charts showing fluorescence data and melt curve data made possible through PCR amplification systems and chips according to the present disclosure. Fluorescence data and melt curve data for a wild-type target within a sample from a system according to the present disclosure is disclosed in FIGS. 12A-12B, and melt curve data for the wild-type target from the system according to the present disclosure is disclosed in FIG. 12C. In particular, FIG. 12C shows that the system and chip according to the present disclosure can provide targeted amplification and detection of a specific wild-type target signal within a sample and, as shown in FIGS. 11A-11C, can distinguish that signal from a specific target SNP signal.

FIG. 13 is an illustration of a flow diagram of an example method 1300 according to the present disclosure. The method 1300 is described herein as implemented using, for example, the systems and chips described for FIGS. 1-12. However, it should be appreciated that the disclosed method 1300 may be implemented using any other suitable system and chip consistent with the teachings presented herein. In addition, although FIG. 13 depicts steps performed in a particular order for purposes of illustration and discussion, the method 1300 described herein is not limited to any particular order or arrangement unless necessary. One skilled in the art, using the disclosure provided herein, will appreciate that various steps of the methods can be omitted, rearranged, combined, and/or adapted in various ways.

As shown at (1302), the method 1300 begins as a PCR sub-process is being completed. In one aspect, the method 1300 does not begin with a completed PCR process, but instead may begin at the completion or near completion of any other sub process necessary for PCR or melt curve analysis. For example, the method 1300 may begin with collection of a sputum sample from a patient. Moreover, the method 1300 may include prefiltering the sputum sample and pre-processing the sputum sample to remove contaminants (food particles, for example, and dead cell lumps). The method 1300 may also include transferring the sputum sample to the input port of a cartridge or the point of entry of a chip, according to the present disclosure.

In one aspect, at (1302), the method 1300 may include the entire (or a significant portion) of a PCR sub-process. For example, the method 1300 may include adding an extraction buffer consisting of GITC and ethanol, for example, in with the sputum sample to lyse the cells in the sample and expose their genetic material. The method 1300 may also include moving the genetic material/sample to interact with dried magnetic beads and slowly moving the combination back and forth across the dried magnetic beads to suspend them in solution. Any DNA exposed during cell lysis sticks to the magnetic beads, and the resultant liquid slug is moved further through a first channel, through a burst valve, and into a larger main channel, for example. Moreover, the method 1300 may include using a magnet to hold the beads in position, and then pushing the surrounding liquid into a waste port. The method 1300 may also include pushing liquid from a well of wash buffer into the main channel to displace any remaining extraction liquid and facilitate rinsing of the beads. Furthermore, the method 1300 may also include pushing the rinse/wash liquid into the waste port, and second rinsing the beads with liquid from a second well of wash buffer to ensure that the beads are free of GITC, ethanol, or any other contaminant that might inhibit PCR or melt curve analysis. The method 1300 may also include driving an elution buffer from a well into the main channel, through a burst valve, and releasing the purified DNA from the magnetic beads into five parallel “PCR channels” containing dried master mix and primers, for example.

As shown at (1304), the method 1300 includes the channel liquid being pushed to a heating region (e.g., such as the serpentine region 916 in FIGS. 9A-9C, which lines up with and is above the heat block 812 of a related system according to the present disclosure). The method 1300 may include holding the channel liquid on the heating region for 90 seconds for example, or for a few (1-10 or 2-5) minutes, for example, for purposes of the PCR sub-process, for example. As shown at (1304), the method 1300 also includes driving the channel liquid back and forth from the heating region to a heating/cool region (such as the serpentine region 914 in FIGS. 9A-9C which lines up with and is above the heat/cool block 811 of a related system according to the present disclosure) and across an optical detection region (such as the optical detection region 912 in FIGS. 9A-9C which is above and in line with the viewing angle of the optical detection unit of a related system according to the present disclosure). The method 1300 may include the optical detection unit of a related system measuring and logging the fluorescence of the channel liquid. Moreover, in one aspect, the method 1300 may include pausing the channel liquid for about 0 seconds to about 1 minute in the heating region and for about 0 seconds to about 1 minutes on the heating/cool region during each of the amplification cycles. In another aspect, the method 1300 may include pausing the channel liquid for about 3 seconds to about 8 seconds in the heating region and for about 3 seconds to about 8 seconds on the heating/cool region during each of the amplification cycles.

As shown at (1306), once the PCR sub-process 1302 is completed, for example, the method 1300 includes moving the channel liquid to the cool region (such as the serpentine region 914 in FIGS. 9A-9C).

As shown at (1308), the method 1300 includes cooling each of the heating region (such as serpentine region 916 in FIGS. 9A-9C) and the cool region (such as the serpentine region 914 in FIGS. 9A-9C) to about 65.0 degrees C.

As shown at (1310), the method 1300 includes moving the channel liquid from the cool region (see 1306) to the heating region and towards/past the optical detection region (such as the optical detection region 912 in FIGS. 9A-9C).

As shown at (1312), the method 1300 includes taking a fluorescence measure via an optical detection unit (such as the photodiode 806 in FIGS. 8A-8F, for example).

As shown at (1314), the method 1300 includes determining, via a processor or any other computing system according to the present disclosure, whether the intensity of the fluorescence measure is greater than a threshold value. If the intensity is less than or equal to the threshold value, then the method 1300 returns to (1312) for further fluorescence measurements and back to (1314) for comparison with the threshold value as the channel liquid is pushed quickly back and forth between the heating region and cool region, for example. In one aspect, the threshold value is preprogrammed and/or remotely updateable. In one aspect, the threshold value is based on loss-of-fluorescence values and measures. In one aspect, the threshold value is based on gain-of-fluorescence values and measures.

As shown at (1316), if the intensity is greater than the threshold value, then the method 1300 includes moving the channel liquid to the cool region.

As shown at (1318), the method 1300 includes increasing the temperature of each of the cool region and the heating region by 0.X degrees per second.

As shown at (1320), the method 1300 includes keeping the channel liquid waiting Y seconds on the cool region. In one aspect, the method 1300 includes pausing the channel liquid for about 0 seconds to about 1 minutes or for about 3 seconds to about 8 seconds (the same or different as called for at (1304)).

As shown at (1322), the method 1300 includes determining, via a processor or any other computing system according to the present disclosure, whether the temperature of the regions is greater than about 95.0 degrees C. If the temperature is less than or equal to about 95.0 degrees C., then the method 1300 returns for: (1) further moving of the channel liquid from the cool region to the heating region and towards/past the optical detection region (1310); (2) further fluorescence measurements (1312); (3) further comparing with the threshold value as the channel liquid is pushed quickly back and forth between the heating region and cool region (1314); further moving of the channel liquid to the cool region if the intensity is greater than the threshold value (1316); further increasing of the temperature of each of the cool region and the heating region by 0.X degrees per second (1318); further holding the channel liquid another Y seconds on the cool region (1320); further determining whether the temperature of the regions is greater than about 95.0 degrees C. (1322); and further continuing the cycle between (1310) and (1322) until the temperature of each of the regions is greater than about 95.0 degrees C.

As shown at (1324), if the temperature is greater than about 95.0 degrees C., then the method 1300 ends and the data is processed.

### IV. Embodiments

Certain implementations of systems and methods consistent with the present disclosure are provided as follows:

Clause 1. A system for identifying a target amplicon for diagnosing tuberculosis and associated antibiotic resistance, comprising: a mechanical instrument; a cartridge assembly, comprising: one or more reservoirs, wherein at least one of the one or more reservoirs contains a wet reagent; a chip, comprising: a fluidic channel holding a liquid slug, comprising an optical detection region between a heating region and a cool region, wherein the mechanical instrument controls a back and forth motion of the liquid slug between the heating region and the cool region; and one or more puncture elements configured to pierce the one or more reservoirs to provide the wet reagent to the chip; and an optical detection unit, comprising: an optical light-emitting element; an optical detector; and a processing unit for performing analysis on the optical detection region of the chip.

Clause 2. The system of clause 1, further comprising at least one heat block, wherein the chip is situated within proximity to the at least one heat block such that the at least one heat block is configured to heat at least the heating region of the fluidic channel of the chip.

Clause 3. The system of clause 1, further comprising a first independent heat block and a second independent heat block, wherein the chip is situated within proximity to the first independent heat block and the second independent heat block such that the first independent heat block is configured to heat at least the heating region of the fluidic channel of the chip and the second independent heat block is configured to heat at least the cool region of the fluidic channel.

Clause 4. The system of clause 1, wherein the optical detection region of the fluidic channel is straight.

Clause 5. The system of clause 1, wherein the heating region of the fluidic channel is a serpentine region.

Clause 6. The system of clause 1, wherein the cool region of the fluidic channel is a serpentine region.

Clause 7. The system of clause 1, wherein the optical detection region of the fluidic channel is straight, and the heating region of the fluidic channel is a serpentine region.

Clause 8. The system of clause 1, wherein the optical detection region of the fluidic channel is straight, and the cool region of the fluidic channel is a serpentine region.

Clause 9. The system of clause 1, wherein the optical detection region of the fluidic channel is straight, the heating region of the fluidic channel is a serpentine region, and the cool region of the fluidic channel is a serpentine region.

Clause 10. The system of clause 1, wherein the mechanical instrument is an overhead drum, wherein the overhead drum is positioned to depress pins onto the chip for fluidic control.

Clause 11. A cartridge and chip for identifying a target amplicon for diagnosing tuberculosis and associated antibiotic resistance, comprising: a cartridge assembly, comprising: one or more reservoirs, wherein at least one of the one or more reservoirs contains a wet reagent; and a chip, comprising: a fluidic channel, comprising an optical detection region between a heating region and a cool region, wherein the fluidic channel is configured to hold a liquid slug that moves back and forth between the heating region and the cool region; and one or more puncture elements configured to pierce the one or more reservoirs to provide the wet reagent to the chip.

Clause 12. The cartridge and chip of clause 11, wherein the optical detection region of the fluidic channel is straight.

Clause 13. The cartridge and chip of clause 11, wherein the heating region of the fluidic channel is a serpentine region.

Clause 14. The cartridge and chip of clause 11, wherein the cool region of the fluidic channel is a serpentine region.

Clause 15. The cartridge and chip of clause 11, wherein the optical detection region of the fluidic channel is straight, and the heating region of the fluidic channel is a serpentine region.

Clause 16. The cartridge and chip of clause 11, wherein the optical detection region of the fluidic channel is straight, and the cool region of the fluidic channel is a serpentine region.

Clause 17. The cartridge and chip of clause 11, wherein the optical detection region of the fluidic channel is straight, the heating region of the fluidic channel is a serpentine region, and the cool region of the fluidic channel is a serpentine region.

Clause 18. The cartridge and chip of clause 11, wherein the cartridge assembly and the chip are oriented such that engaging the cartridge assembly and the chip assembly causes the one or more puncture elements to pierce the one or more reservoirs.

Clause 19. The cartridge and chip of clause 11, wherein the chip further comprises a plurality of superparamagnetic silica-coated beads.

Clause 20: The cartridge and chip of claim 11, wherein the chip further comprises a membrane on a surface of the chip.

Clause 21: The cartridge and chip of claim 11, wherein the cartridge assembly further comprises an input port configured to receive a biological sample, and wherein the cartridge assembly is configured to engage with the chip and transfer the biological sample to the chip.

Clause 22. A method of using a system for identifying a target amplicon for diagnosing tuberculosis and associated antibiotic resistance, comprising: providing a cartridge assembly with a chip, the chip comprising: a fluidic channel comprising an optical detection region between a heating region and a cool region of the fluidic channel, wherein the fluidic channel is configured to hold a liquid slug, and wherein the fluidic channel is configured for the liquid slug to move between the heating region and the cool region; one or more reservoirs containing a wet reagent; and one or more puncture elements configured to pierce the one or more reservoirs of the cartridge assembly to provide the wet reagent to the chip when the cartridge with the chip is assembled and inserted into a mechanical instrument; providing the mechanical instrument, the mechanical instrument comprising: an overhead drum, the overhead drum positioned to depress pins onto the chip for fluidic control of the liquid slug; and an optical detection unit, comprising: an optical light-emitting element; an optical detector; and a processing unit; inserting the cartridge with chip into the mechanical instrument; moving the liquid slug back and forth between the heating region and the cool region through the optical detection region; gradually heating the heating region;

performing, via the processing unit, amplification analysis and on the optical detection region using the optical light-emitting element or the optical detector.

Clause 23: The method of claim 22, further comprising: rotating the overhead drum, with a shaft and a motor, around a central axis; contacting at least one pin with a lobe on the overhead drum; and depressing the at least one pin onto the chip for fluidic control of the liquid slug.

Clause 24: The method of claim 23, further comprising driving the at least one pin in a first direction with the lobe.

Clause 25: The method of claim 24, further comprising moving the at least one pin in a second direction, opposite the first direction, using an elasticity of a membrane of the chip.

Clause 26: The method of claim 23, further comprising sustaining the rotating to contact at least one additional pin with at least one lobe on the overhead drum to depress the at least one additional pin onto the chip.

Clause 27: The method of claim 26, wherein the rotating effectuates depressing of a plurality of pins in a desired timing and sequence onto the chip for fluidic control of a plurality of liquid slugs.

Clause 28: The method of claim 27, further comprising driving the at least one pin in a first direction with the lobe.

Clause 29: The method of claim 28, further comprising moving the at least one pin in a second direction, opposite the first direction, using an elasticity of a membrane of the chip.

Clause 30: The method of claim 29, wherein providing the cartridge assembly with the chip, further comprises providing the chip with a membrane on a surface of the chip over the fluidic channel.

Clause 31: The method of claim 22, further comprising: providing a biological sample to the cartridge assembly; engaging the cartridge assembly with the chip to transfer the biological sample to the chip; and moving the biological sample, via the liquid slug, through the fluidic channel.

Clause 32. A cartridge and chip for identifying a target amplicon for diagnosing tuberculosis and associated antibiotic resistance, comprising: a cartridge assembly, comprising: a plurality of reservoirs, wherein at least one of the plurality of reservoirs contains a wet reagent; and a chip, comprising: five puncture elements configured to pierce at least five reservoirs of the plurality of reservoirs to provide the wet reagent to the chip, and a corresponding fluidic channel for each of the five puncture elements, wherein each corresponding fluidic channel comprises an optical detection region between a heating region and a cool region, and wherein each corresponding fluidic channel is configured to hold a liquid slug that moves back and forth between the heating region and the cool region.

Clause 33. The cartridge and chip of claim 32, wherein the optical detection region of each corresponding fluidic channel is straight, the heating region of each corresponding fluidic channel is a serpentine region, and the cool region of each corresponding fluidic channel is a serpentine region.

Clause 34. The cartridge and chip of claim 32, wherein the cartridge assembly and the chip are oriented such that engaging the cartridge assembly and the chip assembly causes the five puncture elements to pierce at least five reservoirs of the plurality of reservoirs.

Clause 35. The cartridge and chip of claim 32, wherein the chip further comprises a plurality of superparamagnetic silica-coated beads.

Clause 36. The cartridge and chip of claim 32, wherein the chip further comprises a membrane on a surface of the chip.

Clause 37. The cartridge and chip of claim 32, wherein the cartridge assembly further comprises an input port configured to receive a biological sample, and wherein the cartridge assembly is configured to engage with the chip and transfer the biological sample to the chip.

Clause 38. The cartridge and chip of claim 32, wherein each corresponding fluidic channel comprises a corresponding point of entry.

Clause 39. The cartridge and chip of claim 33, wherein the serpentine heating region is upstream of the serpentine cool region.

Clause 40. The cartridge and chip of claim 39, wherein each corresponding fluidic channel is configured to hold the liquid slug that moves back and forth between the serpentine heating region and the serpentine cool region and that is intermittently kept on the optical detection region.

Clause 41. A method of identifying a target amplicon for diagnosing tuberculosis and associated antibiotic resistance using a mechanical instrument comprising an optical detection unit comprising an optical light-emitting element and an optical detector, and using a cartridge assembly with a chip, the chip comprising a fluidic channel comprising an optical detection region between a heating region and a cool region, the cartridge assembly with the chip processed by the mechanical instrument for the method, the method comprising: introducing a liquid slug into a fluidic channel; moving the liquid slug back and forth between a heating region and a cool region through an optical detection region; gradually heating the heating region; performing, via a processing unit, nucleic acid amplification analysis and melt curve analysis on the optical detection region using an optical light-emitting element or an optical detector.

Clause 42. The method of claim 41, further comprising: providing a biological sample to a cartridge assembly; engaging the cartridge assembly with a chip to transfer a biological sample to the chip; and moving the biological sample, via the liquid slug, through the fluidic channel.

Clause 43. The method of claim 42, further comprising pre-filtering the biological sample.

Clause 44. The method of claim 42, further comprising pre-processing the biological sample to remove contaminants.

Clause 45. The method of claim 42, further comprising transferring the biological sample to a point of entry of the chip.

Clause 46. The method of claim 42, wherein the optical detection region of the fluidic channel is straight.

Clause 47. The method of claim 42, wherein the heating region of the fluidic channel is a serpentine region.

Clause 48. The method of claim 42, wherein the cool region of the fluidic channel is a serpentine region.

Clause 49. The method of claim 42, wherein the optical detection region of the fluidic channel is straight, the heating region of the fluidic channel is a serpentine region, and the cool region of the fluidic channel is a serpentine region.

Clause 50. The method of claim 42, wherein moving the liquid slug back and forth between the heating region and the cool region through the optical detection region and performing nucleic acid amplification analysis and melt curve analysis on the optical detection region, further comprises: performing a PCR sub-process; performing a melting sub-process; moving the liquid to the cool region; heating the heating region and the cool region to 65.0 degrees C.; moving the liquid slug to the optical detection region; measuring fluorescence intensity; if intensity of fluorescence is less than or equal to a threshold value, then driving the liquid slug back and forth between the heating region and cool region and measuring fluorescence again.

Clause 51. The method of claim 42, wherein moving the liquid slug back and forth between the heating region and the cool region through the optical detection region and performing nucleic acid amplification analysis and melt curve analysis on the optical detection region, further comprises: performing a PCR sub-process; performing a melting sub-process; moving the liquid to the cool region; heating the heating region and the cool region to 65.0 degrees C.; moving the liquid slug to the optical detection region; measuring fluorescence intensity; if intensity of fluorescence is greater than a threshold value, then moving the liquid slug to the cool region; increasing the temperature of both the heating region and the cool region by a pre-determined degrees C. per second; waiting a predetermined amount of time; measuring the temperature of the liquid slug; if the temperature of the liquid slug is greater than 95.0 degrees C., then end.

Clause 52. The method of claim 42, wherein moving the liquid slug back and forth between the heating region and the cool region through the optical detection region and performing nucleic acid amplification analysis and melt curve analysis on the optical detection region, further comprises: performing a PCR sub-process; performing a melting sub-process; moving the liquid to the cool region; heating the heating region and the cool region to 65.0 degrees C.; moving the liquid slug to the optical detection region; measuring fluorescence intensity; if intensity of fluorescence is greater than a threshold value, then moving the liquid slug to the cool region; increasing the temperature of both the heating region and the cool region by a pre-determined degrees C. per second; waiting a predetermined amount of time; measuring the temperature of the liquid slug; if the temperature of the liquid slug is less than or equal to 95.0 degrees C., then move the liquid slug to the optical detection region; and remeasure fluorescence intensity and determine if remeasured fluorescence intensity is greater than the threshold value.

Clause 53. A method of identifying a target amplicon for diagnosing tuberculosis and associated antibiotic resistance using a mechanical instrument comprising an optical detection unit comprising an optical light-emitting element and an optical detector, and using a cartridge assembly with a chip, the chip comprising a plurality of fluidic channels with each fluidic channel comprising an optical detection region between a heating region and a cool region, the cartridge assembly with the chip processed by the mechanical instrument for the method, the method comprising: introducing a corresponding liquid slug into each of two or more fluidic channels of a plurality of fluidic channels; moving the corresponding liquid slug back and forth between a heating region and a cool region through an optical detection region; gradually heating the heating region of the fluid channels; performing, via a processing unit, nucleic acid amplification analysis and melt curve analysis on the optical detection region using an optical light-emitting element or an optical detector.

Clause 54. The method of claim 53, wherein each fluidic channel comprises a corresponding point of entry.

Clause 55. The method of claim 54, wherein the optical detection region of each fluidic channel is straight, the heating region of each fluidic channel is a serpentine region, and the cool region of each fluidic channel is a serpentine region.

Clause 56. The method of claim 55, wherein the serpentine heating region is upstream of the serpentine cool region.

Clause 57. The method of claim 56, wherein each fluidic channel is configured to hold the corresponding liquid slug that moves back and forth between the serpentine heating region and the serpentine cool region and that is intermittently kept on the optical detection region.

Clause 58. The method of claim 54, further comprising:
providing a biological sample to a cartridge assembly;
engaging the cartridge assembly with a chip to transfer, via the point of entry, the biological sample to the chip; and
moving the biological sample through the fluidic channel.

Clause 59. The method of claim 58, further comprising pre-filtering the biological sample.

Clause 60. The method of claim 58, further comprising pre-processing the biological sample to remove contaminants.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “plurality” means “two or more”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for identifying a target amplicon for diagnosing tuberculosis and associated antibiotic resistance, comprising:
a mechanical instrument;
a cartridge assembly, comprising:
one or more reservoirs, wherein at least one of the one or more reservoirs contains a wet reagent;
a chip, comprising:
a fluidic channel holding a liquid slug, comprising an optical detection region between a heating region and a cool region, wherein the mechanical instrument controls a back and forth motion of the liquid slug between the heating region and the cool region; and
one or more puncture elements configured to pierce the one or more reservoirs to provide the wet reagent to the chip; and
an optical detection unit, comprising:
an optical light-emitting element;
an optical detector; and
a processing unit for performing analysis on the optical detection region of the chip.

2. The system of claim 1, further comprising at least one heat block, wherein the chip is situated within proximity to the at least one heat block such that the at least one heat block is configured to heat at least the heating region of the fluidic channel of the chip.

3. The system of claim 1, further comprising a first independent heat block and a second independent heat block, wherein the chip is situated within proximity to the first independent heat block and the second independent heat block such that the first independent heat block is configured to heat at least the heating region of the fluidic channel of the chip and the second independent heat block is configured to heat at least the cool region of the fluidic channel.

4. The system of claim 1, wherein the optical detection region of the fluidic channel is straight.

5. The system of claim 1, wherein the heating region of the fluidic channel is a serpentine region.

6. The system of claim 1, wherein the cool region of the fluidic channel is a serpentine region.

7. The system of claim 1, wherein the optical detection region of the fluidic channel is straight, and the heating region of the fluidic channel is a serpentine region.

8. The system of claim 1, wherein the optical detection region of the fluidic channel is straight, and the cool region of the fluidic channel is a serpentine region.

9. The system of claim 1, wherein the optical detection region of the fluidic channel is straight, the heating region of the fluidic channel is a serpentine region, and the cool region of the fluidic channel is a serpentine region.

10. The system of claim 1, wherein the mechanical instrument is an overhead drum, wherein the overhead drum is positioned to depress pins onto the chip for fluidic control.

11. A method of using a system for identifying a target amplicon for diagnosing tuberculosis and associated antibiotic resistance, comprising:
providing a cartridge assembly with a chip, the chip comprising:
a fluidic channel comprising an optical detection region between a heating region and a cool region of the fluidic channel, wherein the fluidic channel is configured to hold a liquid slug, and wherein the fluidic channel is configured for the liquid slug to move between the heating region and the cool region;
one or more reservoirs containing a wet reagent; and
one or more puncture elements configured to pierce the one or more reservoirs of the cartridge assembly to provide the wet reagent to the chip when the cartridge with the chip is assembled and inserted into a mechanical instrument;
providing the mechanical instrument, the mechanical instrument comprising:
an overhead drum, the overhead drum positioned to depress pins onto the chip for fluidic control of the liquid slug; and
an optical detection unit, comprising:
an optical light-emitting element;
an optical detector; and
a processing unit;
inserting the cartridge with chip into the mechanical instrument;
moving the liquid slug back and forth between the heating region and the cool region through the optical detection region;
gradually heating the heating region;
performing, via the processing unit, nucleic acid amplification analysis and melt curve analysis on the optical detection region using the optical light-emitting element or the optical detector.

12. The method of claim 11, further comprising:
rotating the overhead drum, with a shaft and a motor, around a central axis;
contacting at least one pin with a lobe on the overhead drum; and
depressing the at least one pin onto the chip for fluidic control of the liquid slug.

13. The method of claim 12, further comprising driving the at least one pin in a first direction with the lobe.

14. The method of claim 13, further comprising moving the at least one pin in a second direction, opposite the first direction, using an elasticity of a membrane of the chip.

15. The method of claim 12, further comprising sustaining the rotating to contact at least one additional pin with at least one lobe on the overhead drum to depress the at least one additional pin onto the chip.

16. The method of claim 15, wherein the rotating effectuates depressing of a plurality of pins in a desired timing and sequence onto the chip for fluidic control of a plurality of liquid slugs.

17. The method of claim 16, further comprising driving the at least one pin in a first direction with the lobe.

18. The method of claim 17, further comprising moving the at least one pin in a second direction, opposite the first direction, using an elasticity of a membrane of the chip.

19. The method of claim 11, wherein providing the cartridge assembly with the chip, further comprises providing the chip with a membrane on a surface of the chip.

20. The method of claim 11, further comprising:
providing a biological sample to the cartridge assembly;
engaging the cartridge assembly with the chip to transfer the biological sample to the chip; and
moving the biological sample, via the liquid slug, through the fluidic channel.
